(19) United States
(12) Patent Application Publication
(10) Pub. No.: US 2026/0097042 A1
(43) Pub. Date: Apr. 9, 2026

(54) COMPOUND EMULSION, PREPARATION METHOD THEREFOR, AND USE THEREOF

(71) Applicant: QILU PHARMACEUTICAL CO., LTD., Jinan (CN)

(72) Inventors: Weiwei Zou, Jinan (CN); Donghai Wang, Jinan (CN); Zhichao Zhou, Jinan (CN); Qingmin Yang, Jinan (CN); Minghui Zhang, Jinan (CN)

(21) Appl. No.: 19/223,047

(22) Filed: May 30, 2025

(30) Foreign Application Priority Data
Oct. 8, 2024 (CN) 202411392722.6
May 23, 2025 (CN) 202510676670.3

(51) Int. Cl. A61K 31/5377; A61K 9/00; A61K 9/107; A61K 31/473; A61K 47/02; A61K 47/10; A61K 47/12; A61K 47/18; A61K 47/22; A61K 47/24; A61K 47/26; A61K 47/44; A61P 1/08

(52) U.S. Cl. CPC A61K 31/5377; A61K 9/0019; A61K 9/107; A61K 31/473; A61K 47/02; A61K 47/10; A61K 47/12; A61K 47/183; A61K 47/22; A61K 47/24; A61K 47/26; A61K 47/44; A61P 1/08

(57) ABSTRACT

The present disclosure provides a compound emulsion for injection of aprepitant and palonosetron hydrochloride. The compound emulsion has good stability and is more convenient and effective in preventing and treating nausea and vomiting, which improves patient medication adherence.

### CROSS-REFERENCE TO RELATED APPLICATIONS

The present application claims the benefit of Chinese Patent Application No. 202411392722.6 filed on Oct. 8, 2024, and the benefit of Chinese Patent Application No. 202510676670.3 filed on May 23, 2025, the contents of which are incorporated herein by reference in their entirety.

### TECHNICAL FIELD

The present disclosure belongs to the field of pharmaceutical preparations, and specifically relates to a compound emulsion for injection of aprepitant and palonosetron hydrochloride, a formulation composition thereof, a preparation method therefor, and a use thereof.

### BACKGROUND

Chemotherapy-induced nausea and vomiting (CINV) is one of the most common adverse reactions during chemotherapy in patients with tumor. A survey based on 2,388 oncology clinicians revealed that 45% of medical staff reported delaying or terminating chemotherapy for patients due to CINV, while 70% considered the ease of administration of medication to the patient as the most important factor. Therefore, timely and effective prevention of CINV is of great significance for improving the quality of life of patients and ensuring the smooth progress of chemotherapy.

Aprepitant is a representative drug among NK₁ inhibitors. It blocks the action sites of substance P by binding to NK₁ receptors, can pass through the blood-brain barrier and occupy NK₁ receptors in the brain, exhibits high selectivity and high affinity, but shows low affinity for NK₂ and NK₃ receptors, resulting in superior efficacy in reducing nausea and vomiting compared to other drugs. The oral preparation of aprepitant was approved by the U.S. FDA in 2003. However, due to its poor water solubility and membrane permeability, aprepitant exhibits poor oral absorption and low bioavailability. In order to improve the shortcomings of oral preparations, Heron Therapeutics developed aprepitant into a fat emulsion injection, which was approved by the FDA for marketing in 2017.

Palonosetron is a second-generation 5-hydroxytryptamine 3 receptor antagonist (5-HT₃RA) with a longer half-life (approximately 40 hours) compared to first-generation 5-HT₃RAs. Its receptor affinity is 30 to 100 times that of the first-generation 5-HT₃RAs, and a single administration dose requires only 0.25 mg. Palonosetron is a highly effective and selective 5-HT₃ receptor blocker that can effectively prevent delayed nausea and vomiting induced by chemotherapy drugs.

Currently, various important guidelines in the field of CINV recommend a triple antiemetic regimen of an NK₁ receptor antagonist+5-HT₃ receptor antagonist+dexamethasone, which is widely used for preventing CINV caused by highly emetogenic chemotherapy and demonstrates significant clinical efficacy. However, this regimen requires administration on different days post-chemotherapy, with complex dosage and route of administration, which brings inconvenience to clinical application. Studies have found that current clinical practices, both domestically and internationally, show unsatisfactory adherence to guidelines: a single-center study in China revealed that adherence to CINV guidelines was only 45% in the acute phase and 7% in the delayed phase, among patients in the single-day chemotherapy group. On the other hand, studies have pointed out that patient non-compliance with medication due to the complexity of multi-drug antiemetic regimens is an important factor for the failure of CINV prevention. Therefore, the development of a convenient and effective antiemetic drug that can improve patient adherence, such as a compound preparation, is of great clinical significance. However, it is explicitly recorded in the precautions of the instruction of aprepitant injection that aprepitant injection should not be mixed with solutions whose physical and chemical compatibility has not been determined. Similarly, it is also recorded in the precautions of the instruction of palonosetron hydrochloride injection that this product should not be mixed with other drugs, and the infusion line should be flushed with normal saline before and after infusion. Early related reports also recorded that palonosetron hydrochloride is stable in a low pH range under acidic conditions, with optimal stability at pH 5. However, aprepitant injection is stable at relatively high pH under alkaline conditions, with a pH of 7.5-9.0, preferably 8-9. It is explicitly recorded that pH should be controlled during manufacturing, and a decrease in pH may indicate chemical degradation. Additionally, studies have shown that palonosetron hydrochloride is prone to oxidation to generate impurity A, while aprepitant injection contains a large amount of excipients such as soybean oil and phospholipids, which are unsaturated fatty acids prone to oxidation to generate peroxides. Therefore, during the development of compound preparations, how to find a formulation system that can make both stable is a difficult problem that needs to be solved urgently.

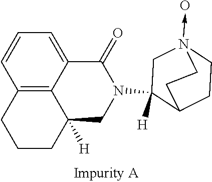 [SMILES: O=C1c2cccc3c2[C@H](CCC3)CN1[C@@H]1C[N+]2([O-])CCC1CC2]

### BRIEF SUMMARY OF THE INVENTION

In view of the complexity of administration in multidrug antiemetic regimens and poor patient adherence in the prior art, the present disclosure provides a stable compound emulsion for injection of aprepitant and palonosetron hydrochloride, a preparation method therefor, and a use thereof.

Specifically,

A first aspect of the present disclosure provides a compound emulsion for injection comprising aprepitant, palonosetron hydrochloride, and a pharmaceutically acceptable carrier, wherein the pharmaceutically acceptable carrier comprises an antioxidant.

In some embodiments of the present disclosure, the pharmaceutically acceptable carrier in the compound emulsion comprises an antioxidant and a metal chelating agent.

In some embodiments of the present disclosure, the pharmaceutically acceptable carrier in the compound emulsion comprises one or a mixture of two or more of an antioxidant, a metal chelating agent, an emulsifier, a co-emulsifier, an oil for injection, an osmotic pressure adjuster, a pH adjuster, and water for injection, preferably comprising an antioxidant, a metal chelating agent, an emulsifier, a co-emulsifier, an oil for injection, an osmotic pressure adjuster, a pH adjuster, and water for injection.

In some embodiments of the present disclosure, the compound emulsion consists of aprepitant, palonosetron hydrochloride, and a pharmaceutically acceptable carrier, wherein the pharmaceutically acceptable carrier is an antioxidant, a metal chelating agent, an emulsifier, a co-emulsifier, an oil for injection, an osmotic pressure adjuster, a pH adjuster, and water for injection.

In some embodiments of the present disclosure, the antioxidant in the compound emulsion is selected from the group consisting of one or a mixture of two or more of sodium bisulfite, anhydrous sodium sulfite, sodium metabisulfite, sodium thiosulfate, ascorbic acid, cysteine, citric acid, sodium citrate, and vitamin C.

In some embodiments of the present disclosure, the antioxidant in the compound emulsion is a mixture of sodium bisulfite and anhydrous sodium sulfite.

In some embodiments of the present disclosure, the mass percentage of the antioxidant in the compound emulsion is 0.01% to 0.783%, preferably, the mass percentage of the antioxidant is 0.02%.

In some embodiments of the present disclosure, the percentage content of the antioxidant in the compound emulsion is 0.01% to 0.783%, preferably, the percentage content of the antioxidant is 0.02%. In some embodiments of the present disclosure, the antioxidant in the compound emulsion is a mixture of sodium bisulfite and anhydrous sodium sulfite, and the weight ratio of sodium bisulfite to anhydrous sodium sulfite is 1:1, preferably, the antioxidant in the compound emulsion is a mixture of sodium bisulfite and anhydrous sodium sulfite, the weight ratio of sodium bisulfite to anhydrous sodium sulfite is 1:1, the mass percentage of sodium bisulfite is 0.01%, and the mass percentage of anhydrous sodium sulfite is 0.01%.

In some embodiments of the present disclosure, the antioxidant in the compound emulsion is a mixture of sodium bisulfite and anhydrous sodium sulfite, the weight ratio of sodium bisulfite to anhydrous sodium sulfite is 1:1, the percentage content of sodium bisulfite is 0.01%, and the percentage content of anhydrous sodium sulfite is 0.01%.

In some embodiments of the present disclosure, the metal chelating agent in the compound emulsion is selected from the group consisting of one or a mixture of two or more of edetic acid, disodium edetate, tetrasodium edetate, disodium nitrilotriacetate, pentetic acid, citric acid, tartaric acid, and gluconic acid.

In some embodiments of the present disclosure, the metal chelating agent in the compound emulsion is disodium edetate.

In some embodiments of the present disclosure, the mass percentage of the metal chelating agent in the compound emulsion is 0.004% to 0.055%, preferably, the mass percentage of the metal chelating agent is 0.004% to 0.008%, more preferably, the mass percentage of the metal chelating agent is 0.005%.

In some embodiments of the present disclosure, the percentage content of the metal chelating agent in the compound emulsion is 0.004% to 0.055%, preferably, the percentage content of the metal chelating agent is 0.004% to 0.008%, more preferably, the percentage content of the metal chelating agent is 0.005%.

In some embodiments of the present disclosure, the emulsifier in the compound emulsion is selected from the group consisting of one or a mixture of two or more of egg yolk lecithin, soybean lecithin, and polyethylene glycol 15-hydroxystearate.

In some embodiments of the present disclosure, the emulsifier in the compound emulsion is egg yolk lecithin.

In some embodiments of the present disclosure, the percentage content of the emulsifier in the compound emulsion is 7% to 20%; for example, 14.4%.

In some embodiments of the present disclosure, the co-emulsifier in the compound emulsion is selected from the group consisting of one or a mixture of two or more of anhydrous ethanol, oleic acid, sodium oleate, poloxamer, and polysorbate 80.

In some embodiments of the present disclosure, the co-emulsifier in the compound emulsion is anhydrous ethanol.

In some embodiments of the present disclosure, the mass percentage of the co-emulsifier in the compound emulsion is 1% to 4%; for example, 2.8%. In some embodiments of the present disclosure, the oil for injection in the compound emulsion is selected from the group consisting of one or a mixture of two or more of soybean oil, olive oil, safflower oil, tea seed oil, peanut oil, fish oil, castor oil, medium-chain triglycerides, polyoxyethylene castor oil, rapeseed oil, corn oil, and sesame oil.

In some embodiments of the present disclosure, the oil for injection in the compound emulsion is soybean oil.

In some embodiments of the present disclosure, the percentage content of the oil for injection in the compound emulsion is 5% to 15%; for example, 9.4%.

In some embodiments of the present disclosure, the osmotic pressure adjuster in the compound emulsion is selected from the group consisting of one or a mixture of two or more of sucrose, glycerol, mannitol, and glucose.

In some embodiments of the present disclosure, the osmotic pressure adjuster in the compound emulsion is sucrose.

In some embodiments of the present disclosure, the percentage content of the osmotic pressure adjuster in the compound emulsion is 3% to 10%; for example, 5.5%.

In some embodiments of the present disclosure, the pH adjuster in the compound emulsion is selected from the group consisting of one or a mixture of two or more of sodium oleate, oleic acid, sodium hydroxide, and hydrochloric acid.

In some embodiments of the present disclosure, the pH adjuster in the compound emulsion is sodium oleate.

In some embodiments of the present disclosure, the pH adjuster in the compound emulsion is used to adjust the pH of the compound emulsion to 7.0 to 9.0.

In some embodiments of the present disclosure, the compound emulsion is supplemented with water for injection to make up the remaining volume.

In some embodiments of the present disclosure, the mass ratio of aprepitant to palonosetron hydrochloride (calculated as palonosetron) in the compound emulsion is (400 to 600):1; for example, 520:1.

In some embodiments of the present disclosure, the concentration of aprepitant is 5 to 10 mg/mL, for example, 7.2 mg/mL.

In some embodiments of the present disclosure, the concentration of palonosetron hydrochloride (calculated as palonosetron) is 0.005 to 0.02 mg/mL, for example, 0.014 mg/mL.

In some embodiments of the present disclosure, the compound emulsion comprises 130 mg of aprepitant per unit preparation and 0.25 mg of palonosetron hydrochloride (calculated as palonosetron) per unit preparation.

In some embodiments of the present disclosure, the compound emulsion is 18 mL per unit preparation.

In some embodiments of the present disclosure, the compound emulsion has a pH value of 7.0 to 9.0; preferably, the compound emulsion has a pH value of 7.0, 7.5, 8.0, 8.5, or 9.0.

In some embodiments of the present disclosure, the compound emulsion has a viscosity of 5 mPa·s to 25 mPa·s, for example, 9 mPa·s to 15 mPa·s, for another example, 9 mPa·s, 11 mPa·s, 13 mPa·s, or 15 mPa·s.

In some embodiments of the present disclosure, the compound emulsion has an average particle size of 60 to 100 nm, for example, 70 to 90 nm.

In some embodiments of the present disclosure, 90% of emulsion particles in the compound emulsion has a light intensity particle size D90 (90% of the droplets in the emulsion have a particle size smaller than this value) of less than 200 nm, for example, 130 nm to 180 nm.

In some embodiments of the present disclosure, the compound emulsion has a Zeta potential of −10 mV to −70 mV, for example, −30 mV to −70 mV, for another example, −50.7 mV, −53.4 mV, −39.1 mV, −56.5 mV, −50.1 mV, or −57.2 mV.

In some embodiments of the present disclosure, the compound emulsion has an anisidine value of ≤25.0, for example, ≤15, for another example, 6 to 7; for a further example, 6.5.

In some embodiments of the present disclosure, the compound emulsion has a peroxide value of ≤1.0 mL, for example, 0.05.

In some embodiments of the present disclosure, the compound emulsion has a palonosetron impurity A

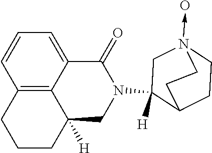 [SMILES: O=C1c2cccc3c2[C@H](CCC3)CN1[C@@H]1C[N+]2([O-])CCC1CC2]

of ≤1.0% (% represents mass percentage), for example, 0.09% to 0.39%, for another example, 0.09%, 0.13%, 0.20%, 0.27%, or 0.39%.

In some embodiments of the present disclosure, other individual impurities in the compound emulsion are ≤1.0% (% represents mass percentage), for example, no other individual impurities are detected, wherein the individual impurities refer to impurities related to palonosetron.

In some embodiments of the present disclosure, the total impurities in the compound emulsion are ≤2.0% (% represents mass percentage), for example, 0.09% to 0.39%, for another example, 0.09%, 0.13%, 0.20%, 0.27%, or 0.39%, wherein the total impurities refer to impurities related to palonosetron.

In some embodiments of the present disclosure, the compound emulsion may be used in combination with a glucocorticoid drug.

In some embodiments of the present disclosure, the compound emulsion may be used in combination with a glucocorticoid drug, wherein the glucocorticoid is dexamethasone or prednisone.

In some embodiments of the present disclosure, the components and their parts by mass in the above compound emulsion are as follows:

| | | |
|---|---|---|
| Palonosetron hydrochloride | 1 | part |
| (calculated as palonosetron) | | |
| Aprepitant | 520 | parts |
| Emulsifier | 10400 | parts |
| Co-emulsifier | 2000 | parts |
| Oil for injection | 6800 | parts |
| pH adjuster | 400 | parts |
| Osmotic pressure adjuster | 4000 | parts |
| Metal chelating agent | 3 to 40 | parts |
| Antioxidant | 7 to 35 | parts |
| Water for injection | 48000 | parts |

In some embodiments of the present disclosure, the components and their parts by mass in the above compound emulsion are as follows:

| | | |
|---|---|---|
| Palonosetron hydrochloride | 1 | part |
| (calculated as palonosetron) | | |
| Aprepitant | 520 | parts |
| Emulsifier | 10400 | parts |
| Co-emulsifier | 2000 | parts |
| Oil for injection | 6800 | parts |
| pH adjuster | 400 | parts |
| Osmotic pressure adjuster | 4000 | parts |
| Metal chelating agent | 3.6 | parts |
| Antioxidant | 14.4 | parts |
| Water for injection | 48000 | parts |

In some embodiments of the present disclosure, the components and their parts by mass in the above compound emulsion are as follows:

| | | |
|---|---|---|
| Palonosetron hydrochloride | 1 | part |
| (calculated as palonosetron) | | |
| Aprepitant | 520 | parts |
| Egg yolk lecithin | 10400 | parts |
| Anhydrous ethanol | 2000 | parts |
| Soybean oil | 6800 | parts |
| Sodium oleate | 400 | parts |
| Sucrose | 4000 | parts |
| Disodium edetate | 3.6 | parts |
| Sodium bisulfite | 7.2 | parts |
| Anhydrous sodium sulfite | 7.2 | parts |
| Water for injection | 48000 | parts |

In some embodiments of the present disclosure, the composition and percentage content (% W/V) of each component in the above compound emulsion are as follows:

| Names of raw materials | Percentage |
|---|---|
| and excipients | content (%, w/v) |
| Aprepitant | 0.722 |
| Palonosetron hydrochloride | 0.00139 |
| (calculated as palonosetron) | |
| Egg yolk lecithin | 14.444 |
| Anhydrous ethanol | 2.778 |
| Soybean oil | 9.444 |
| Sodium oleate | 0.556 |
| Sucrose | 5.556 |
| Disodium edetate | 0.005 |
| Sodium bisulfite | 0.01 |
| Anhydrous sodium sulfite | 0.01 |
| Water for injection | 66.667 |

wherein the compound emulsion comprises 130 mg of aprepitant per unit preparation and 0.25 mg of palonosetron hydrochloride (calculated as palonosetron) per unit preparation. The above compound emulsion is 18 mL per unit preparation.

A second aspect of the present disclosure provides a preparation method for the above compound emulsion, comprising the following steps:
(1) mixing aprepitant, an emulsifier, and a co-emulsifier with an oil for injection to prepare an oil phase;
(2) mixing water for injection, an osmotic pressure adjuster, a pH adjuster, and a metal chelating agent to prepare an aqueous phase;
(3) mixing the oil phase with the aqueous phase, and performing high-speed shear dispersion to prepare a crude emulsion;
(4) homogenizing the crude emulsion through a microfluidizer under high pressure to prepare a final emulsion;
(5) adding an antioxidant and palonosetron hydrochloride to the final emulsion, and stirring and dissolving;
(6) sterilizing, nitrogen-purging, and filling a drug emulsion;
wherein aprepitant, palonosetron hydrochloride, the antioxidant, the metal chelating agent, the emulsifier, the co-emulsifier, the oil for injection, the osmotic pressure adjuster, and the pH adjuster are as defined above.

A third aspect of the present disclosure provides a use of the above compound emulsion:

The present disclosure provides use of the compound emulsion in the manufacture of a medicament for preventing acute and/or delayed nausea and vomiting occurring during initial or repeated treatment with a highly emetogenic chemotherapy drug (HEC).

The present disclosure provides the compound emulsion for use in preventing acute and/or delayed nausea and/or vomiting occurring during initial or repeated treatment with a moderately emetogenic chemotherapy drug (MEC).

The compound emulsion is preferably administered prior to the administration of the highly emetogenic chemotherapy drug (HEC) or the moderately emetogenic chemotherapy drug (MEC) in a subject, to effectively prevent acute or delayed nausea or vomiting occurring during initial or repeated treatment with a highly emetogenic chemotherapy drug (HEC) or a moderately emetogenic chemotherapy drug (MEC).

### Technical Effect

The compound emulsion provided in the present disclosure exhibits good stability, so that aripiprazole in the system can be encapsulated within emulsified oil droplet particles and remain stable under preparation and storage conditions without demulsification or phase separation; at the same time, the palonosetron hydrochloride can also be stable without significant increase in the representative oxidative impurity A. Furthermore, the compound emulsion of the present disclosure has a remarkable effect in preventing/treating nausea and vomiting.

### Description and Definition

In the present disclosure, the percentage content (%, W/V) represents the mass (in grams) of solute contained per 100 mL of solution.

Emulsions are thermodynamically unstable heterogeneous dispersion systems, often experiencing phenomena such as phase separation, flocculation, phase inversion, cracking, and rancidity during preparation or storage.

(1) Phase separation: refers to the phenomenon where dispersed phase particles float or sink during the storage of the emulsion, which is a reversible process. The primary reason is the density difference between the dispersed phase and the continuous phase.

Common methods to slow down the rate of phase separation are: reducing the particle size of the emulsion droplets, increasing the viscosity of the continuous phase, and decreasing the density difference between the dispersed phase and the continuous phase.
(2) Flocculation: The phenomenon where dispersed droplets in an emulsion aggregate into loose clusters that can be restored to a uniform emulsion upon shaking is termed as the flocculation of the emulsion, which is a precursor to emulsion cracking. The cause is attributed to a reduction in zeta potential.
(3) Phase inversion: refers to the change in the type of emulsion, which is caused by adding another substance to the emulsion, altering the properties of the emulsifier.
(4) Cracking: The separation of an emulsion into oil and aqueous phases is referred to as emulsion cracking, ultimately resulting in visible free oil on the surface of the emulsion, which is an irreversible process.
(5) Rancidity: The phenomenon where the oil, emulsifiers, etc., in an emulsion undergo deterioration due to external factors (light, heat, air, etc.) and microorganisms is referred to as rancidity. Additionally, unsaturated fatty acids in the structure of soybean oil and phospholipid excipients are highly susceptible to oxidation, forming peroxides. These peroxides are quite unstable and can further decompose into small molecular compounds such as aldehydes, ketones, and acids, increasing the anisidine value of the system. The addition of antioxidants and preservatives can prevent or delay rancidity.

The types and amounts of excipients in the emulsion, drug properties, zeta potential of the system, pH value, residual oxygen content, dissolved oxygen content, preparation process, packaging materials, storage conditions, etc. all influence the physicochemical stability of the emulsion.

Data show that palonosetron hydrochloride is prone to oxidative degradation catalyzed by metal ions in water. Forced degradation test studies have shown that palonosetron hydrochloride is sensitive to oxidation, readily undergoing oxidative reactions in this emulsion system to form impurity A.

Unless otherwise specified, the “unit preparation” in the present disclosure refers to a minimum preparation form of a drug, such as a capsule, a tablet, a vial, or a patch.

Unless otherwise specified, “used in combination” in the present disclosure means that two drugs are respectively contained in preparation units of different specifications and may be administered simultaneously, sequentially, or at intervals.

### DETAILED DESCRIPTION OF THE INVENTION

In order to better illustrate the present disclosure, the preferred embodiments will be described in detail below with reference to examples. It should be understood that the following examples are provided for illustrative purposes only and are not intended to limit the scope of the present disclosure. Simple improvements to the present disclosure made under the premise of the technical solutions of the present disclosure fall within the protection scope of the present disclosure.

### 1. Formulation Design

Based on the specifications and clinical dosages of single-ingredient preparations of aprepitant injection and palonosetron hydrochloride injection, the developed specification of this compound emulsion is determined to be 18 mL: 130 mg of aprepitant and 0.25 mg of palonosetron. With reference to the formulation compositions of commercially available single-ingredient aprepitant injection emulsions domestically and internationally, a basic formulation composition was preliminarily proposed, namely, aprepitant and palonosetron hydrochloride as active ingredients, egg yolk lecithin as an emulsifier, anhydrous ethanol as a co-emulsifier, soybean oil as an oil-phase solvent, sodium oleate as a pH adjuster, sucrose as an osmotic pressure adjuster, and water for injection as an aqueous-phase solvent.

The preliminarily determined basic formulation composition is shown in Table 1.

TABLE 1
Basic formulation composition of compound emulsion

| Names of raw materials | Basic formulation | | |
|---|---|---|---|
| and excipients | Amount per vial | | Function |
| Aprepitant | 130 | mg | Active ingredient |
| Palonosetron hydrochloride | 0.25 | mg | Active ingredient |
| (calculated as palonosetron) | | | |
| Egg yolk lecithin | 2.6 | g | Emulsifier |
| Anhydrous ethanol | 0.5 | g | Co-emulsifier |
| Soybean oil | 1.7 | g | Oil-phase solvent |
| Sodium oleate | 0.1 | g | pH adjuster |
| Sucrose | 1 | g | Osmotic pressure |
| | | | adjuster |
| Water for injection | 12 | g | Aqueous-phase |
| | | | solvent |

### 2. Formulation Screening and Optimization

### (1) Determination of the Amount of Disodium Edetate

Based on the basic formulation, with other components fixed, samples with different amounts of disodium edetate (0%, 0.005%, 0.05%) were prepared to investigate the effect of the amount of disodium edetate on the related substance II during stability storage of this product.

TABLE 2
Screening of the amount of disodium edetate in formulation composition

| | Disodium edetate | | | Disodium edetate | | | Disodium edetate | | |
|---|---|---|---|---|---|---|---|---|---|
| | 0% | | | 0.005% | | | 0.05% | | |
| Names of raw | | | Percentage | | | Percentage | | | Percentage |
| materials and | Amount | | content | Amount | | content | Amount | | content |
| excipients | per vial | | (%, w/v) | per vial | | (%, w/v) | per vial | | (%, w/v) |
| Aprepitant | 130 | mg | 0.722 | 130 | mg | 0.722 | 130 | mg | 0.722 |
| Palonosetron | 0.25 | mg | 0.00139 | 0.25 | mg | 0.00139 | 0.25 | mg | 0.00139 |
| hydrochloride | | | | | | | | | |
| (calculated as | | | | | | | | | |
| palonosetron) | | | | | | | | | |
| Egg yolk lecithin | 2.6 | g | 14.444 | 2.6 | g | 14.444 | 2.6 | g | 14.444 |
| Anhydrous | 0.5 | g | 2.778 | 0.5 | g | 2.778 | 0.5 | g | 2.778 |
| ethanol | | | | | | | | | |
| Soybean oil | 1.7 | g | 9.444 | 1.7 | g | 9.444 | 1.7 | g | 9.444 |
| Sodium oleate | 0.1 | g | 0.556 | 0.1 | g | 0.556 | 0.1 | g | 0.556 |
| Sucrose | 1 | g | 5.556 | 1 | g | 5.556 | 1 | g | 5.556 |
| Disodium | 0 | | 0 | 0.9 | mg | 0.005 | 9 | mg | 0.05 |
| edetate | | | | | | | | | |
| Water for | 12 | g | 66.667 | 12 | g | 66.667 | 12 | g | 66.667 |
| injection | | | | | | | | | |

TABLE 3
Effect of the amount of disodium edetate on related substances II

| | | Related substances II | | |
|---|---|---|---|---|
| Amount of | Storage | | Other | Total |
| disodium | conditions/ | Palonosetron | individual | impurities |
| edetate | time | impurity A (%) | impurity (%) | (%) |
| Test standard | | ≤1.0% | ≤1.0% | ≤2.0% |
| 0% | 0 days | 0.16 | N.D. | 0.16 |
| | 2 to 8° C. for 1 | 0.62 | N.D. | 0.62 |
| | month | | | |
| | 25° C./60% for 1 | 1.2 | N.D. | 1.2 |
| | month | | | |
| 0.005% | 0 days | 0.08 | N.D. | 0.08 |
| | 2 to 8° C. for 1 | 0.36 | N.D. | 0.36 |
| | month | | | |
| | 25° C./60% for 1 | 1.4 | N.D. | 1.4 |
| | month | | | |
| 0.05% | 0 days | 0.08 | N.D. | 0.08 |
| | 2 to 8° C. for 1 | 0.34 | N.D. | 0.34 |
| | month | | | |
| | 25° C./60% for 1 | 0.69 | N.D. | 0.69 |
| | month | | | |

“N.D.” stands for not detected.

The results show that the formulation without disodium edetate exhibited significant increase in palonosetron impurity A (oxidation impurity) after storage at 2 to 8° C. for 1 month, whereas the formulations with 0.005% and 0.05% amount of disodium edetate demonstrated smaller increments in impurity A with no substantial difference between the two. Under storage conditions of 25° C./60% for 1 month, all three batches of samples showed significant increases in impurity A, and the impurity A exceeded the limit in both the formulation without disodium edetate and the formulation with 0.005% amount of disodium edetate. This indicates that adding a certain amount of disodium edetate on the formulation can inhibit the growth of impurity A to a certain extent, while the antioxidant effect is not significant under accelerated conditions, and it is necessary to add an antioxidant in the formulation.

Detection method for related substance II:

Octylsilyl-bonded silica gel was used as a filler (Agilent ZORBAX RX-C8, 4.6 mm×250 mm, 5 μm or a chromatographic column with equivalent performance); with acetonitrile-water-trifluoroacetic acid (300:700:0.67) as mobile phase A and tetrahydrofuran-water (80:20) as mobile phase B, gradient elution was performed according to the following table [a Ghost Trap DS pre-column (specifications: 7.6 mm×30 mm) was installed between a gradient mixer and an autosampler]; the flow rate was 1.0 mL per minute; the column temperature was 30° C.; the detection wavelength was 210 nm; the injection volume was 100 μL.

TABLE 4
Chromatographic conditions for the detection
method of related substance II

| Time (min) | Mobile phase A (%) | Mobile phase B (%) |
|---|---|---|
| 0 | 100 | 0 |
| 15 | 100 | 0 |
| 16 | 0 | 100 |
| 26 | 0 | 100 |
| 27 | 100 | 0 |
| 37 | 100 | 0 |

### (2) Screening of Antioxidant Types and Amounts

Based on the proposed basic formulation composition, samples containing different types and amounts of antioxidants were prepared. These samples were stored at 30° C./65% for 30 days to evaluate key physicochemical indicators and investigate the stability of different formulations.

TABLE 5
Screening of antioxidant types and amounts
in formulation composition (1)

| | Amount per vial | | | | | |
|---|---|---|---|---|---|---|
| Names of raw materials | 0.1% (w/v) | | 0.03% (w/v) | | 0.5% (w/v) | |
| and excipients | Vitamin C | | Citric acid | | Tocopherol | |
| Aprepitant | 130 | mg | 130 | mg | 130 | mg |
| Palonosetron hydrochloride | 0.25 | mg | 0.25 | mg | 0.25 | mg |
| (calculated as palonosetron) | | | | | | |
| Egg yolk lecithin | 2.6 | g | 2.6 | g | 2.6 | g |
| Anhydrous ethanol | 0.5 | g | 0.5 | g | 0.5 | g |
| Soybean oil | 1.7 | g | 1.7 | g | 1.7 | g |
| Sodium oleate | 0.1 | g | 0.1 | g | 0.1 | g |
| Sucrose | 1 | g | 1 | g | 1 | g |
| Disodium edetate | 0.9 | mg | 0.9 | mg | 0.9 | mg |
| Vitamin C | 18 | mg | — | | — | |
| Citric acid | — | | 5.4 | mg | — | |
| Tocopherol | — | | — | | 90 | mg |
| Water for injection | 12 | g | 12 | g | 12 | g |

TABLE 6
Screening of antioxidant types and amounts in formulation composition (2)

| | Amount per vial | | | | | | | |
|---|---|---|---|---|---|---|---|---|
| | | | | | | | 0.01% (w/v) Sodium | |
| Names of raw | 0.01% (w/v) | | 0.01% (w/v) | | 0.05% (w/v) | | bisulfite + 0.01% | |
| materials and | Anhydrous | | Sodium | | Sodium | | (w/v) Anhydrous | |
| excipients | sodium sulfite | | bisulfite | | bisulfite | | sodium sulfite | |
| Aprepitant | 130 | mg | 130 | mg | 130 | mg | 130 | mg |
| Palonosetron | 0.25 | mg | 0.25 | mg | 0.25 | mg | 0.25 | mg |
| hydrochloride | | | | | | | | |
| (calculated as | | | | | | | | |
| palonosetron) | | | | | | | | |
| Egg yolk lecithin | 2.6 | g | 2.6 | g | 2.6 | g | 2.6 | g |
| Anhydrous ethanol | 0.5 | g | 0.5 | g | 0.5 | g | 0.5 | g |
| Soybean oil | 1.7 | g | 1.7 | g | 1.7 | g | 1.7 | g |
| Sodium oleate | 0.1 | g | 0.1 | g | 0.1 | g | 0.1 | g |
| Sucrose | 1 | g | 1 | g | 1 | g | 1 | g |
| Disodium edetate | 0.9 | mg | 0.9 | mg | 0.9 | mg | 0.9 | mg |
| Sodium bisulfite | — | | 1.8 | mg | 9 | mg | 1.8 | mg |
| Anhydrous sodium | 1.8 | mg | — | | — | | 1.8 | mg |
| sulfite | | | | | | | | |
| Water for injection | 12 | g | 12 | g | 12 | g | 12 | g |

TABLE 7
Effect of antioxidant type and amount on stability

| | | Test item | | | | | | | | | |
|---|---|---|---|---|---|---|---|---|---|---|---|
| | | | | Related substances II | | | Particle size | | | | |
| | | | | | Other | | Average | | | | |
| | | | | Palonosetron | individual | Total | particle | | Zeta | Peroxide | |
| Anti- | Investigation | | pH | impurity | impurity | impurities | size | D₉₀ | potential | value | Anisidine |
| oxidant | time | Appearance | value | A (%) | (%) | (%) | (nm) | (nm) | (mV) | (mL) | value |
| Test standard | | Off- | 7.0 to | ≤1.0% | ≤1.0% | ≤2.0% | 60 to | <200 nm | −10 mV | ≤1.0 mL | ≤25.0 |
| | | white | 9.0 | | | | 100 nm | | to −70 | | |
| | | to | | | | | | | mV | | |
| | | yellowish- | | | | | | | | | |
| | | brown | | | | | | | | | |
| | | milky | | | | | | | | | |
| | | liquid | | | | | | | | | |
| 0.1% | 0 days | Pale | 7.6 | 0.03 | N.D. | 0.03 | 85 | 172 | −43.9 | 0.05 | 4.5 |
| Vitamin C | | yellow | | | | | | | | | |
| | | milky | | | | | | | | | |
| | | liquid | | | | | | | | | |
| | 30 days | Yellowish- | 7.1 | 0.04 | N.D. | 0.0 | 121 | 224 | −45.2 | 0.05 | /# |
| | | brown | | | | | | | | | |
| | | milky | | | | | | | | | |
| | | liquid | | | | | | | | | |
| 0.03% | 0 days | Pale | 7.8 | 0.02 | N.D. | 0.02 | 87 | 175 | −49.0 | 1.25 | 4.3 |
| Citric acid | | yellow | | | | | | | | | |
| | | milky | | | | | | | | | |
| | | liquid | | | | | | | | | |
| | 30 days | Yellow | 7.6 | 0.45 | N.D. | 0.45 | 96 | 178 | −51.7 | 0.65 | 5.3 |
| | | milky | | | | | | | | | |
| | | liquid | | | | | | | | | |
| 0.5% | 0 days | Pale | 8.4 | 0.11 | N.D. | 0.11 | 87 | 170 | −51.9 | 2.65 | 3.6 |
| Tocopherol | | yellow | | | | | | | | | |
| | | milky | | | | | | | | | |
| | | liquid | | | | | | | | | |
| | 30 days | Pale | 8.0 | 1.9 | N.D. | 1.9 | 86 | 163 | −54.4 | 1.32 | 5.6 |
| | | yellow | | | | | | | | | |
| | | milky | | | | | | | | | |
| | | liquid | | | | | | | | | |
| 0.01% | 0 days | Pale | 8.5 | 0.16 | N.D. | 0.16 | 86 | 180 | −49.5 | 0.05 | 6.2 |
| Anhydrous | | yellow | | | | | | | | | |
| sodium | | milky | | | | | | | | | |
| sulfite | | liquid | | | | | | | | | |
| | 30 days | Yellow | 8.0 | 0.88 | N.D. | 0.88 | 90 | 172 | −51.4 | 0.80 | 6.8 |
| | | milky | | | | | | | | | |
| | | liquid | | | | | | | | | |
| 0.01% | 0 days | Pale | 8.4 | 0.17 | N.D. | 0.17 | 87 | 166 | −55.2 | 0.05 | 10.7 |
| Sodium | | yellow | | | | | | | | | |
| bisulfite | | milky | | | | | | | | | |
| | | liquid | | | | | | | | | |
| | 30 days | Pale | 8.0 | 0.56 | N.D. | 0.56 | 90 | 177 | −47.6 | 0.05 | 5.1 |
| | | yellow | | | | | | | | | |
| | | milky | | | | | | | | | |
| | | liquid | | | | | | | | | |
| 0.05% | 0 days | Pale | 7.9 | 0.03 | N.D. | 0.03 | 87 | 176 | −53.9 | 0.05 | 8.6 |
| Sodium | | yellow | | | | | | | | | |
| bisulfite | | milky | | | | | | | | | |
| | | liquid | | | | | | | | | |
| | 30 days | Pale | 7.8 | 0.05 | N.D. | 0.05 | 96 | 186 | −49.1 | 0.05 | 10.0 |
| | | yellow | | | | | | | | | |
| | | milky | | | | | | | | | |
| | | liquid | | | | | | | | | |
| 0.01% | 0 days | Pale | 8.4 | 0.18 | N.D. | 0.18 | 88 | 174 | −57.2 | 0.05 | 6.5 |
| Sodium | | yellow | | | | | | | | | |
| bisulfite + | | milky | | | | | | | | | |
| 0.01% | | liquid | | | | | | | | | |
| Anhydrous | 30 days | Pale | 8.0 | 0.29 | N.D. | 0.29 | 88 | 175 | −53.8 | 0.05 | 6.2 |
| sodium | | yellow | | | | | | | | | |
| sulfite | | milky | | | | | | | | | |
| | | liquid | | | | | | | | | |

Note:
“N.D.” stands for not detected; the anisidine value of 0.1% vitamin C batch at 30 days could not be determined due to sample color interference, hence no test results were obtained.

### Results Analysis

The formulation with 0.1% vitamin C as the antioxidant showed a palonosetron impurity A level comparable to that at the initial time point after 30 days at 30° C./65%; however, significant changes were observed in appearance, the particle size increased significantly, with both the average particle size and D90 exceeding the upper limit, and the pH value approaching the lower limit, indicating poor stability;

The formulation with 0.03% citric acid as the antioxidant showed a peroxide value exceeding the limit at the initial time point. After 30 days at 30° C./65%, changes in appearance were observed, with significant increases in palonosetron impurity A and particle size, indicating poor stability;

The formulation with 0.5% tocopherol as the antioxidant showed a peroxide value far exceeding the upper limit at the initial time point. After 30 days at 30° C./65%, palonosetron impurity A increased significantly and far exceeded the upper limit, indicating poor stability;

The formulation with 0.01% anhydrous sodium sulfite as the antioxidant showed altered appearance after 30 days at 30° C./65%, with slight pH decrease, significant increases in palonosetron impurity A and peroxide value, and slight particle size growth, indicating poor stability;

The formulation with 0.01% sodium bisulfite as the antioxidant showed slight pH decrease after 30 days at 30° C./65%, with significant increase in palonosetron impurity A and slight particle size growth, indicating poor stability;

The formulation with 0.05% sodium bisulfite as the antioxidant showed the palonosetron impurity A comparable to that at the initial time point after 30 days at 30° C./65%, but showed significant particle size growth, indicating poor stability;

The formulation with 0.01% sodium bisulfite+0.01% anhydrous sodium sulfite as the antioxidants showed slight increase in palonosetron impurity A and slight pH decrease after 30 days at 30° C./65% compared to those at the initial time point. Other key physicochemical indicators (such as appearance, particle size, Zeta potential, peroxide value, and anisidine value) remained comparable to those at the initial time point, with all evaluated indicators meeting requirements. This demonstrates that this formulation has good stability and is significantly better than other formulations with other antioxidants. The analysis suggests that the combined use of two antioxidants, anhydrous sodium sulfite and sodium bisulfite, can produce a synergistic effect, thereby enhancing the antioxidative performance;

Therefore, the antioxidants in the formulation of this product are preferably sodium bisulfite and anhydrous sodium sulfite, each with an amount of 0.01%.

### (3) pH Value Investigation

pH value is an important quality indicator of the preparation and may have a certain impact on the key quality attributes of this product. The impact of different pH values on the key physicochemical indicators of the final product was investigated. The Formulation composition is shown in Table 8, and the test results are shown in Table 9.

TABLE 8
Sample formulation for pH value investigation

| Names of raw materials | | | Percentage content |
|---|---|---|---|
| and excipients | Amount | | (%, w/v) |
| Aprepitant | 130 | mg | 0.722 |
| Palonosetron hydrochloride | 0.25 | mg | 0.00139 |
| (calculated as palonosetron) | | | |
| Egg yolk lecithin | 2.6 | g | 14.444 |
| Anhydrous ethanol | 0.5 | g | 2.778 |
| Soybean oil | 1.7 | g | 9.444 |
| Sodium oleate | 0.1 | g | 0.556 |
| Sucrose | 1 | g | 5.556 |
| Disodium edetate | 0.9 | mg | 0.005 |
| Sodium bisulfite | 1.8 | mg | 0.01 |
| Anhydrous sodium sulfite | 1.8 | mg | 0.01 |
| Water for injection | 12 | g | 66.667 |

1) Preparation of aqueous phase: Sucrose, sodium oleate, and disodium edetate were weighed, added to water for injection, and heated in the range of 40 to 80° C. for dissolution.
2) Preparation of oil phase: Aprepitant, egg yolk lecithin, soybean oil, and anhydrous ethanol were weighed, and heated in the range of 40 to 80° C. for dissolution.
3) Preparation of crude emulsion by combining phases: The oil and aqueous phases were mixed and subjected to high-speed shear dispersion in the range of 40 to 80° C.
4) Preparation of final emulsion: The crude emulsion was homogenized using a microfluidizer under conditions of 20 to 70° C. and 3000 psi to 25000 psi to obtain the final emulsion.
5) Dissolution of antioxidant and palonosetron hydrochloride raw materials: Sodium bisulfite, anhydrous sodium sulfite, and palonosetron hydrochloride were added to the final emulsion and stirred for dissolution.
6) Adjustment of pH value and sterile filtration: The pH value was adjusted to 6.5, 7.0, 7.5, 8.0, 8.5, 9.0, or 9.5 using dilute hydrochloric acid and 1M sodium hydroxide solution, respectively, followed by sterile filtration of the bulk solution.

The samples at different pH values were filled into vials, followed by nitrogen purging, stoppering, and capping. Key physicochemical indicators (appearance, viscosity, emulsion particle, Zeta potential, related substance II, free fatty acids, lysophosphatidylcholine, and lysophosphatidylethanolamine) were subsequently tested.

TABLE 9
Effect of pH value on key quality attributes of products

| | | pH | | | | | | |
|---|---|---|---|---|---|---|---|---|
| Test standard | | 6.5 | 7.0 | 7.5 | 8.0 | 8.5 | 9.0 | 9.5 |
| Appearance | Off-white to | Pale | Pale | Pale | Pale | Pale | Pale | Pale |
| | yellowish- | yellow | yellow | yellow | yellow | yellow | yellow | yellow |
| | brown milky | milky | milky | milky | milky | milky | milky | milky |
| | liquid | liquid | liquid | liquid | liquid | liquid | liquid | liquid |
| Viscosity | 5 to 25 cP | 9 | 9 | 11 | 13 | 15 | 15 | 15 |
| (mPa · s) | (mPa · s) | | | | | | | |
| Emulsion | The average | 91 | 78 | 77 | 78 | 78 | 78 | 77 |
| particle (nm) | particle size | | | | | | | |
| | should be 60 | | | | | | | |
| | to 100 nm | | | | | | | |
| | The light | 169 | 152 | 141 | 148 | 155 | 147 | 144 |
| | intensity | | | | | | | |
| | particle size | | | | | | | |
| | of 90% of the | | | | | | | |
| | emulsion | | | | | | | |
| | particles | | | | | | | |
| | should be less | | | | | | | |
| | than 200 nm | | | | | | | |
| Zeta | −10 mV to −70 | −39.0 | −50.7 | −53.4 | −39.1 | −56.5 | −50.1 | −55.4 |
| potential | mV | | | | | | | |
| (mV) | | | | | | | | |
| Related | Palonosetron | 0.06 | 0.09 | 0.13 | 0.20 | 0.27 | 0.39 | 0.52 |
| substance II | impurity A ≤1.0% | | | | | | | |
| (%) | Other | 0.16 | 0.13 | 0.12 | 0.13 | 0.10 | 0.10 | 0.09 |
| | individual | | | | | | | |
| | impurities ≤1.0% | | | | | | | |
| | Total | 0.06 | 0.09 | 0.13 | 0.20 | 0.27 | 0.39 | 0.52 |
| | impurities ≤2.0% | | | | | | | |
| Free fatty | The volume | Complies | Complies | Complies | Complies | Complies | Complies | Complies |
| acid | (in mL) of | | | | | | | |
| | sodium | | | | | | | |
| | hydroxide | | | | | | | |
| | titrant (0.01 | | | | | | | |
| | mol/L) | | | | | | | |
| | consumed by | | | | | | | |
| | the test | | | | | | | |
| | solution shall | | | | | | | |
| | not be greater | | | | | | | |
| | than the | | | | | | | |
| | volume (in | | | | | | | |
| | mL) of | | | | | | | |
| | sodium | | | | | | | |
| | hydroxide | | | | | | | |
| | titrant (0.01 | | | | | | | |
| | mol/L) | | | | | | | |
| | consumed by | | | | | | | |
| | the control | | | | | | | |
| | solution | | | | | | | |
| Lysophos- | Each 1 mL of | LPE: 0.86 | LPE: 0.81 | LPE: 0.81 | LPE: 0.82 | LPE: 0.76 | LPE: 0.79 | LPE: 0.84 |
| phatidyl- | this product | LPC: 3.0 | LPC: 3.0 | LPC: 3.0 | LPC: 3.0 | LPC: 3.0 | LPC: 3.1 | LPC: 3.4 |
| ethanolamine | shall contain | | | | | | | |
| and | not more than | | | | | | | |
| lysophos- | 7.2 mg of | | | | | | | |
| phatidyl- | lysophos- | | | | | | | |
| choline | phatidyl- | | | | | | | |
| (mg/mL) | ethanolamine | | | | | | | |
| | and not more | | | | | | | |
| | than 24.0 mg | | | | | | | |
| | of lysophos- | | | | | | | |
| | phatidyl- | | | | | | | |
| | choline | | | | | | | |

Note:
The ignoring threshold for other individual impurities is 0.3%, i.e., other individual impurities <0.3% are not included in the total impurities. For the above batches of samples, all other individual impurities were below the ignoring threshold, hence only the known impurity palonosetron impurity A was calculated when calculating the total impurities.

The results show that within the pH range of 6.5 to 9.5, the viscosity slightly decreases with the decrease of pH value; palonosetron impurity A significantly increases with the increase of pH value, reaching the highest level at pH 9.5, exceeding 0.5%; the pH value has a slight effect on the emulsion particles of the product, with the largest particle size at pH 6.5 and no significant difference in particle size between pH 7.0 and 9.5; the pH value has no significant effect on free fatty acids, lysophosphatidylethanolamine (LPE), and lysophosphatidylcholine (LPC). Based on the above results, the pH limit for this product is determined to be 7.0 to 9.0 after comprehensive consideration.

### 3. Pharmacodynamic Comparison Experiment

### 1. Experimental Purpose

To evaluate the antiemetic effect of the compound emulsion of the present disclosure on cisplatin-induced vomiting model in ferrets by observing and recording the latency and frequency of retching and vomiting induced by cisplatin in ferrets, while comparing the pharmacodynamic differences between the compound emulsion of the present disclosure and the control single agent at the same dose.

### 2. Experimental Materials

Animal acclimation and grouping: Male ferrets aged 6 months, 1.3 to 2.0 kg. Two animals per cage. After acclimating in the animal facility for at least one week, the animals were randomly grouped based on their body weight.

TABLE 10
Source of materials

| | Source | |
|---|---|---|
| Compound name | (manufacturer/supplier) | Batch No. |
| 0.9% Sodium chloride | Anhui Fengyuan | 1122101202 |
| injection (normal saline) | Pharmaceutical Co., Ltd. | |
| Cisplatin | Anhui Zesheng Technology | C0EORRET |
| | Co., Ltd. | |
| Aprepitant injection (trade | Heron Therapeutics Inc | 83481 |
| name: CINVANTI) | | |
| Palonosetron hydrochloride | LEE'S PHARM | 43000172 |
| injection (trade name: | | |
| ALOXI) | | |
| Compound emulsion of the | The formulation shown | / |
| present disclosure | in Table 8 of the | |
| | present disclosure | |

### 3. Experimental Procedures

### 3.1. Grouping

Prior to experimental testing, the animals were acclimatized in the animal facility for at least one week and randomly grouped based on body weight.

TABLE 11
Experimental grouping

| | Group | | | Number of |
|---|---|---|---|---|
| Group | No. | Dosage | Route of administration | animals (n) |
| Vehicle | 1 | NA | Intraperitoneal administration | n = 8 |
| control group | | | (0.9% sodium chloride injection) + | |
| | | | intravenous injection (0.9% | |
| | | | sodium chloride injection) | |
| Cisplatin | 2 | 10 | Intraperitoneal administration | n = 8 |
| group | | mg/kg | (cisplatin) + intravenous injection | |
| | | | (0.9% sodium chloride injection) | |
| Aprepitant | 3 | 0.2 | Intraperitoneal administration | n = 8 |
| group | | mg/kg | (cisplatin) + intravenous injection | |
| | | | (0.1 mg/mL aprepitant) | |
| Palonosetron | 4 | 0.0004 | Intraperitoneal administration | n = 8 |
| group | | mg/kg | (cisplatin) + intravenous injection | |
| | | | (0.0002 mg/mL palonosetron) | |
| Low-dose | 5 | 0.1002 | Intraperitoneal administration | n = 8 |
| compound | | mg/kg | (cisplatin) + intravenous injection | |
| emulsion | | | (0.0501 mg/mL compound | |
| group of the | | | emulsion of the present disclosure) | |
| present | | | | |
| disclosure | | | | |
| High-dose | 6 | 0.2004 | Intraperitoneal administration | n = 8 |
| compound | | mg/kg | (cisplatin) + intravenous injection | |
| emulsion | | | (0.1002 mg/mL compound | |
| group of the | | | emulsion of the present disclosure) | |
| present | | | | |
| disclosure | | | | |

### 3.2. Administration and Observation

The body weight of the animals was measured. Ten minutes prior to cisplatin administration, the vehicle control group, cisplatin group, aprepitant group, palonosetron group, low-dose compound emulsion group of the present disclosure, and high-dose compound emulsion group of the present disclosure were administered with normal saline, normal saline, aprepitant, palonosetron, the low-dose compound emulsion of the present disclosure, and the high-dose compound emulsion of the present disclosure, respectively. The administration volume was 2 mL/kg.

Ten minutes after administration, all groups of animals were intraperitoneally injected with cisplatin at a dose of 10 mg/kg, with an administration volume of 5 mL/kg.

The latency and number of retching and vomiting episodes in ferrets within 4 hours after cisplatin induction were observed and recorded.
1) Retching latency: Timing was initiated after the administration of cisplatin to the animal, and the time of the first occurrence of retching (without vomitus) was recorded.
2) Vomiting latency: Timing was initiated after the administration of cisplatin to the animal, and the time of the first occurrence of vomiting (with vomitus) was recorded.
3) Number of retching episodes: Timing was initiated after the administration of cisplatin to the animal, and the number of retching episodes (without vomitus) was recorded.
4) Number of vomiting episodes: Timing was initiated after the administration of cisplatin to the animal, and the number of vomiting episodes (with vomitus) was recorded.

### 3.3. Animal Care

Upon completion of the experiment, the animals were euthanized promptly.

### 3.4. Data Collection and Analysis

Data were collected. Data were analyzed statistically, and a P-value less than 0.05 was considered statistically significant. The specific analytical methods were as follows:

The data from each group were tested for normal distribution and homogeneity of variance (F-test). If the data followed a normal distribution and exhibited homogeneity of variance, an unpaired t-test was used to compare the differences between two groups of data, or one/two-way ANOVA was used to compare the differences among multiple groups of data. If the data followed a normal distribution but at least one group of data did not meet the homogeneity of variance assumption, Welch's t-test was used to compare and analyze the differences between two groups of data, or a nonparametric test was used to compare the differences among multiple groups of data. If the data did not follow a normal distribution, the Mann-Whitney test was used to analyze the differences between two groups of data, or the Kruskal-Wallis test was used to analyze the differences among multiple groups of data.

### 4. Experimental Results

### 4.1. Body Weight and Grouping

All animals were randomly grouped (n=8) before administration, with no significant differences in body weight observed among the ferrets in each group. During the experiment, no significant differences in body weight were observed among the animals in each group.

TABLE 12
Body weight of ferrets on the day of random grouping and administration

| | | | | | | Low-dose | High-dose |
|---|---|---|---|---|---|---|---|
| | | | | | | compound | compound |
| | | | | | | emulsion | emulsion |
| | | Vehicle | | | | group of | group of |
| | | control | Cisplatin | Aprepitant | Palonosetron | the present | the present |
| | | group | group | group | group | disclosure | disclosure |
| Body weight at | Mean | 1.59 | 1.57 | 1.57 | 1.57 | 1.57 | 1.57 |
| grouping (kg) | SEM | 0.052 | 0.037 | 0.033 | 0.031 | 0.029 | 0.030 |
| Body weight at | Mean | 1.60 | 1.58 | 1.57 | 1.55 | 1.56 | 1.56 |
| administration (kg) | SEM | 0.057 | 0.035 | 0.030 | 0.031 | 0.029 | 0.034 |

### 4.2. Observation of Antiemetic Effect

The results of this study showed that ferrets (cisplatin group) without antiemetic intervention exhibited retching at 70±8.6 min and vomiting at 74±7.9 min after cisplatin administration, with 164±25.3 retching episodes and 15±3.5 vomiting episodes within 4 hours.

Compared with the cisplatin group: administration of aprepitant (0.2 mg/kg) had little impact on the retching latency in ferrets (72±3.6 min), prevented vomiting in one ferret, and slightly prolonged the vomiting latency (101±20.5 min), but overall showed no significant difference from the cisplatin group. Aprepitant (0.2 mg/kg) could significantly reduce (p< 0.05) the number of retching (73±10.9 episodes) and vomiting (5±1.1 episodes) episodes in ferrets within 4 hours. Administration of palonosetron (0.0004 mg/kg) prevented vomiting and retching in one ferret in this group within 4 hours, but the retching and vomiting latencies, as well as the number of retching and vomiting episodes in most ferrets, were similar to those in the cisplatin group. Overall statistical analysis of various vomiting indicators for this compound showed no significant difference from the cisplatin group (99±24.3 min, 97±24.5 min, 121±26.9 episodes, and 9±2.5 episodes, respectively). Administration of a low dose of the compound emulsion of the present disclosure (0.1002 mg/kg) could slightly prolong the retching and vomiting latencies (83±4.3 min and 129±24.6 min, respectively) in cisplatin-induced ferrets, but without statistical significance; the number of retching episodes was significantly reduced (99±12.4 episodes, p<0.05); and no vomiting was observed in two ferrets in this group within 4 hours (4±1.0 episodes, p<0.05). Administration of a high dose of the compound emulsion of the present disclosure (0.2004 mg/kg) could slightly prolong the retching latency (93±5.5 min, p>0.05) in cisplatin-induced ferrets, and no vomiting was observed in five ferrets in this group within 4 hours (204±19.3 min, p<0.05); the number of retching and vomiting episodes was significantly reduced (34±6.8 and 1±0.6 episodes, respectively, p<0.05). At 0.2004 mg/kg, the pharmacodynamic activity of the test compound—the compound emulsion of the present disclosure-showed no significant difference in retching latency compared to the single agents (p>0.05). However, in terms of vomiting latency, the compound emulsion of the present disclosure exhibited superior pharmacodynamic activity to both aprepitant and palonosetron (p-values for the compound emulsion of the present disclosure v.s. aprepitant and palonosetron: 0.0015 and 0.0010, respectively). For the number of retching episodes (p-values for the compound emulsion of the present disclosure v.s. aprepitant and palonosetron: 0.3509 and 0.0030, respectively) and the number of vomiting episodes (p-values for the compound emulsion of the present disclosure v.s. aprepitant and palonosetron: 0.5456 and 0.0223, respectively), the compound emulsion of the present disclosure showed no statistical difference in the pharmacodynamic activity from aprepitant. However, the compound emulsion of the present disclosure showed the numbers of retching and vomiting episodes (34 episodes, 1 episode) lower than those observed with aprepitant (73 episodes, 5 episodes), and superior to those with palonosetron (121 episodes, 9 episodes).

TABLE 13
Retching latency in each group of ferrets

| | | | | | | Low-dose | High-dose |
|---|---|---|---|---|---|---|---|
| | | | | | | compound | compound |
| | | | | | | emulsion | emulsion |
| | | Vehicle | | | | group of the | group of the |
| | | control | Cisplatin | Aprepitant | Palonosetron | present | present |
| | | group | group | group | group | disclosure | disclosure |
| Latency of retching | Mean | 240 | 70 | 72 | 99 | 83 | 93 |
| (min) | SEM | 0.0 | 8.6 | 3.6 | 24.3 | 4.3 | 5.5 |

| | | | | | | |
|---|---|---|---|---|---|---|
| P (v.s. cisplatin group) | <0.0001 | / | 0.9998 | 0.2193 | 0.8630 | 0.4414 |
| P (v.s. high-dose compound | / | / | 0.5376 | 0.9916 | 0.9530 | / |
| emulsion group of the | | | | | | |
| present disclosure) | | | | | | |

Note:
The vomiting latency of animals without observed vomiting within 4 hours after cisplatin administration was recorded as 240 min.

TABLE 14
Vomiting latency in each group of ferrets

| | | | | | | Low-dose | High-dose |
|---|---|---|---|---|---|---|---|
| | | | | | | compound | compound |
| | | | | | | emulsion | emulsion |
| | | Vehicle | | | | group of the | group of the |
| | | control | Cisplatin | Aprepitant | Palonosetron | present | present |
| | | group | group | group | group | disclosure | disclosure |
| Latency of vomiting | Mean | 240 | 74 | 101 | 97 | 129 | 204 |
| (min) | SEM | 0.0 | 7.9 | 20.5 | 24.5 | 24.6 | 19.3 |

| | | | | | | |
|---|---|---|---|---|---|---|
| P (v.s. cisplatin group) | <0.0001 | / | 0.7543 | 0.8377 | 0.1489 | <0.0001 |
| P (v.s. high-dose compound | / | / | 0.0015 | 0.0010 | 0.0291 | / |
| emulsion group of the | | | | | | |
| present disclosure) | | | | | | |

Note:
The vomiting latency of animals without observed vomiting within 4 hours after cisplatin administration was recorded as 240 min.

TABLE 15
Number of retching episodes in each group of ferrets

| | | | | | | Low-dose | High-dose |
|---|---|---|---|---|---|---|---|
| | | | | | | compound | compound |
| | | | | | | emulsion | emulsion |
| | | Vehicle | | | | group of the | group of the |
| | | control | Cisplatin | Aprepitant | Palonosetron | present | present |
| | | group | group | group | group | disclosure | disclosure |
| Number of | Mean | 0 | 164 | 73 | 121 | 99 | 34 |
| retching episodes | SEM | 0.0 | 25.3 | 10.9 | 26.9 | 12.4 | 6.8 |
| (episodes) | | | | | | | |

| | | | | | | |
|---|---|---|---|---|---|---|
| P (v.s. cisplatin group) | <0.0001 | / | 0.0018 | 0.2646 | 0.0356 | <0.0001 |
| P (v.s. high-dose compound | / | / | 0.3509 | 0.0030 | 0.0374 | / |
| emulsion group of the | | | | | | |
| present disclosure) | | | | | | |

TABLE 16
Number of vomiting episodes in each group of ferrets

| | | | | | | Low-dose | High-dose |
|---|---|---|---|---|---|---|---|
| | | | | | | compound | compound |
| | | Vehicle | | | | emulsion group | emulsion group |
| | | control | Cisplatin | Aprepitant | Palonosetron | of the present | of the present |
| | | group | group | group | group | disclosure | disclosure |
| Number of | Mean | 0 | 15 | 5 | 9 | 4 | 1 |
| vomiting episodes | SEM | 0.0 | 3.5 | 1.1 | 2.5 | 1.0 | 0.6 |
| (episodes) | | | | | | | |

| | | | | | | |
|---|---|---|---|---|---|---|
| P (v.s. cisplatin group) | <0.0001 | / | 0.0016 | 0.1059 | 0.0005 | <0.0001 |
| P (v.s. high-dose compound | / | / | 0.5456 | 0.0223 | 0.8062 | / |
| emulsion group of the | | | | | | |
| present disclosure) | | | | | | |

TABLE 17
Number of retching and vomiting episodes in each group of ferrets

| | | | | | | Low-dose | High-dose |
|---|---|---|---|---|---|---|---|
| | | | | | | compound | compound |
| | | | | | | emulsion | emulsion |
| | | Vehicle | | | | group of the | group of the |
| | | control | Cisplatin | Aprepitant | Palonosetron | present | present |
| | | group | group | group | group | disclosure | disclosure |
| Number of | Mean | 0 | 179 | 77 | 130 | 102 | 35 |
| retching and | SEM | 0.0 | 27.9 | 11.7 | 29.1 | 13.0 | 7.2 |
| vomiting episodes | | | | | | | |
| (episodes) | | | | | | | |

| | | | | | | |
|---|---|---|---|---|---|---|
| P (v.s. cisplatin group) | <0.0001 | / | 0.0014 | 0.2252 | 0.0205 | <0.0001 |
| P (v.s. high-dose compound | / | / | 0.3470 | 0.0029 | 0.0498 | / |
| emulsion group of the | | | | | | |
| present disclosure) | | | | | | |

### CONCLUSION AND DISCUSSION

In summary, under the conditions of the present experiment, the test compound—the compound emulsion of the present disclosure—can dose-dependently inhibit the number of cisplatin-induced retching and vomiting episodes in ferrets within the dose range of 0.1002 and 0.2004 mg/kg, in a dose-dependent manner. Compared with the model group, aprepitant can significantly reduce the number of retching and vomiting episodes in ferrets, but the average number of retching and vomiting episodes are higher than those in the 0.2004 mg/kg group of the compound emulsion of the present disclosure. In addition, palonosetron has little effect on the various vomiting-related indicators observed in this experiment, and its efficacy is significantly weaker than that of the 0.2004 mg/kg group of the compound emulsion of the present disclosure.

1. A compound emulsion for injection comprising aprepitant, palonosetron hydrochloride, and a pharmaceutically acceptable carrier, wherein the pharmaceutically acceptable carrier comprises an antioxidant.

2. The compound emulsion for injection according to claim 1, wherein the pharmaceutically acceptable carrier further comprises a metal chelating agent.

3. The compound emulsion for injection according to claim 2, wherein the pharmaceutically acceptable carrier further comprises one or more of an emulsifier, a co-emulsifier, an oil for injection, an osmotic pressure adjuster, a pH adjuster, and water for injection.

4. The compound emulsion for injection according to claim 1, wherein the antioxidant is selected from the group consisting of one or a mixture of two or more of sodium bisulfite, anhydrous sodium sulfite, sodium metabisulfite, sodium thiosulfate, ascorbic acid, cysteine, citric acid, sodium citrate, and vitamin C.

5. The compound emulsion for injection according to claim 1, wherein the antioxidant is a mixture of sodium bisulfite and anhydrous sodium sulfite, and the weight ratio of sodium bisulfite to anhydrous sodium sulfite is 1:1.

6. The compound emulsion for injection according to claim 1, wherein the percentage content of the antioxidant in the compound emulsion is 0.01% to 0.783%.

7. The compound emulsion for injection according to claim 1, wherein the antioxidant is a mixture of sodium bisulfite and anhydrous sodium sulfite, the weight ratio of sodium bisulfite to anhydrous sodium sulfite is 1:1, the percentage content of sodium bisulfite is 0.01%, and the percentage content of anhydrous sodium sulfite is 0.01%.

8. The compound emulsion for injection according to claim 2, wherein the metal chelating agent is selected from the group consisting of one or a mixture of two or more of edetic acid, disodium edetate, tetrasodium edetate, disodium nitrilotriacetate, pentetic acid, citric acid, tartaric acid, and gluconic acid.

9. The compound emulsion for injection according to claim 2, wherein the percentage content of the metal chelating agent in the compound emulsion is 0.004% to 0.055%.

10. The compound emulsion for injection according to claim 3, which satisfies one or more of the following conditions:
1) the emulsifier is selected from the group consisting of one or a mixture of two or more of egg yolk lecithin, soybean lecithin, and polyethylene glycol 15-hydroxystearate;
2) the co-emulsifier is selected from the group consisting of one or a mixture of two or more of anhydrous ethanol, oleic acid, sodium oleate, poloxamer, and polysorbate 80;
3) the oil for injection is selected from the group consisting of one or a mixture of two or more of soybean oil, olive oil, safflower oil, tea seed oil, peanut oil, fish oil, castor oil, medium-chain triglycerides, polyoxyethylene castor oil, rapeseed oil, corn oil, and sesame oil;
4) the osmotic pressure adjuster is selected from the group consisting of one or a mixture of two or more of sucrose, glycerol, mannitol, and glucose;
5) the pH adjuster is selected from the group consisting of one or a mixture of two or more of sodium oleate, oleic acid, sodium hydroxide, and hydrochloric acid.

11. The compound emulsion for injection according to claim 3, which satisfies one or more of the following conditions:
1) the percentage content of the emulsifier in the compound emulsion is 7% to 20%;
2) the percentage content of the co-emulsifier in the compound emulsion is 1% to 4%;
3) the percentage content of the oil for injection in the compound emulsion is 5% to 15%;
4) the percentage content of the osmotic pressure adjuster in the compound emulsion is 3% to 10%;
5) the concentration of aprepitant is 5 to 10 mg/mL;
6) the concentration of palonosetron hydrochloride (calculated as palonosetron) is 0.005 to 0.02 mg/mL.

12. The compound emulsion for injection according to claim 1, wherein the mass ratio of aprepitant to palonosetron hydrochloride (calculated as palonosetron) is (400 to 600):1.

13. The compound emulsion for injection according to claim 1, wherein the compound emulsion for injection comprises 130 mg of aprepitant per unit preparation and 0.25 mg of palonosetron hydrochloride (calculated as palonosetron) per unit preparation.

14. The compound emulsion for injection according to claim 1, having a pH value of 7.0 to 9.0.

15. The compound emulsion for injection according to claim 1, which satisfies one or more of the following conditions:
1) the compound emulsion has a viscosity of 5 mPa·s to 25 mPa·s;
2) the compound emulsion has an average particle size of 60 to 100 nm;
3) 90% of emulsion particles in the compound emulsion should have a light intensity particle size D90 of less than 200 nm;
4) the compound emulsion has a Zeta potential of −10 mV to −70 mV;
5) the compound emulsion has an anisidine value of ≤25.0;
6) the compound emulsion has a peroxide value of ≤1.0 mL.

16. The compound emulsion for injection according to claim 1, wherein the compound emulsion has a palonosetron impurity A of ≤1.0%.

17. The compound emulsion for injection according to claim 1, wherein a total amount of impurities related to palonosetron in the compound emulsion is ≤2.0%.

18. The compound emulsion for injection according to claim 1, which may be used in combination with a glucocorticoid drug.

19. A preparation method for a compound emulsion for injection, comprising the following steps:
(1) mixing aprepitant, an emulsifier, and a co-emulsifier with an oil for injection to prepare an oil phase;
(2) mixing water for injection, an osmotic pressure adjuster, a pH adjuster, and a metal chelating agent to prepare an aqueous phase;
(3) mixing the oil phase with the aqueous phase, and performing high-speed shear dispersion to prepare a crude emulsion;
(4) homogenizing the crude emulsion through a microfluidizer under high pressure to prepare a final emulsion;
(5) adding an antioxidant and palonosetron hydrochloride to the final emulsion, and stirring and dissolving;
(6) sterilizing, nitrogen-purging, and filling a drug emulsion;
wherein aprepitant, palonosetron hydrochloride, the antioxidant, the metal chelating agent, the emulsifier, the co-emulsifier, the oil for injection, the osmotic pressure adjuster, and the pH adjuster are as defined in claim 3.

20. A method for preventing acute or delayed nausea or vomiting occurring during initial or repeated treatment with a highly emetogenic chemotherapy drug (HEC) or a moderately emetogenic chemotherapy drug (MEC) in a subject in need thereof, comprising administering a therapeutically effective amount of the compound emulsion for injection as defined in claim 1.
